(19) United States
(12) Patent Application Publication
(10) Pub. No.: US 2005/0216012 A1
(43) Pub. Date: Sep. 29, 2005

(54) Bone pin

(75) Inventor: Hans Rainer Willmen, Grevenbroich (DE)

Correspondence Address: OSTROLENK FABER GERB & SOFFEN, 1180 AVENUE OF THE AMERICAS, NEW YORK, NY, 100368403, US

(73) Assignees: HANS RAINER WILLMEN, GREVENBROICH (DE); THOMAS GAUSEPOHL, D-51109 KOLN (DE)

(21) Appl. No.: 10/518,190

(22) PCT Filed: Feb. 21, 2004
(86) PCT No.: PCT/EP04/01728

§ 371 Date: Dec. 16, 2004

(30) Foreign Application Priority Data
Apr. 9, 2003 (DE) 203 05 713.9

(51) Int. Cl. A61F 2/30; A61B 17/60

(52) U.S. Cl. 606/72; 606/61; 606/104

(57) ABSTRACT

The subject of the invention is a bone dowel for inserting a screw, for example for mutual fixing of bone fragments by means of an osteosynthesis plate, in which a gently conical dowel jacket has a through-hole of uniform cross section along its length, and the dowel jacket is interrupted along a continuous generating line by a longitudinal slit, by which means the dowel jacket acquires a continuously C-shaped cross section, and in which a limiting head flange in the form of a countersunk head is formed integrally on the head part of the dowel, and the head part of the dowel jacket is provided, in the area of the longitudinal slit, with a bevel whose width decreases from the head flange toward the dowel jacket. On its outside, the dowel jacket has annular ribs between which there extend longitudinal ribs that are offset in the circumferential direction. The dowel base is rounded in a dome shape.

The invention relates to a bone dowel which can be inserted into a bore in a bone and into which a screw, made of stainless steel or absorbable material for example, can be screwed in order to ensure a reliable, permanent anchoring in the bone. Bone dowels of this kind are used in various surgical techniques. One application is the use of such dowels for fixing bone fragments by means of an osteosynthesis plate, as is described in DE 101 07 201. Bone dowels of this kind can also be used for anchoring tendons in a bone channel, for example in order to stabilize joints after tearing of a capsule/ligament, as is described in detail in DE 100 35 610 A1.

Bone dowels are also used for fixing a thread with which tissue can be secured on a bone. Such a dowel is described in EP 0 502 509 A1.

DE 35 09 417 A1 describes an arrangement for assisting in osteosynthesis in bone surgery, in which a bone dowel for insertion of a screw is provided, a circular cylindrical dowel jacket having a through-hole of uniform cross section along its length, and the dowel jacket being interrupted along a generating line by a longitudinal slit. This longitudinal slit divides the lower portion of the dowel in such a way that two separate dowel branches are formed.

EP 0 340 159 A1 describes an expansion dowel for cementless anchoring of bone implants, in which a hollow body of an expansion dowel for anchoring implants in bone is designed with a polygonal configuration along its outer circumference.

DE 34 17 923 A1 describes an endoprosthesis and a dowel for anchoring in a material having bone-like strength. The endoprosthesis consists of a hollow anchoring part which can be spread open and into whose cavity an expansion body can be inserted, by which means the anchoring part inserted into a bore in the bone is clamped firmly on the walls of the bore in said bone and is thus anchored cementlessly in the bone.

It is an object of the invention to make available a bone dowel which can be used for numerous applications in surgery and ensures a reliable hold. There is an increasing need for this in osteoporotic bone caused by hormonal changes in the female skeleton and by age-related bone degeneration in the elderly. In the specific case of osteoporosis, when osteosynthesis is performed, it can happen that screws are overtightened, so that, for example, metal plates are unable to be securely anchored, making it necessary to switch to other surgical techniques.

The object set is achieved by the totality of the features set out in patent claim 1.

Further expedient embodiments are set out in dependent claims 2 through 10.

The dowel can be made of absorbable material in a manner known per se, in which case the screw, fixed on the wall of the bore in the bone, can remain in the body, or it can be surgically removed.

The bone dowel according to the invention satisfies all the requirements to be met in the specialized field in question, and no similar model can be found in general dowel technology.

Illustrative embodiments of the invention are described below with reference to the drawing, in which:

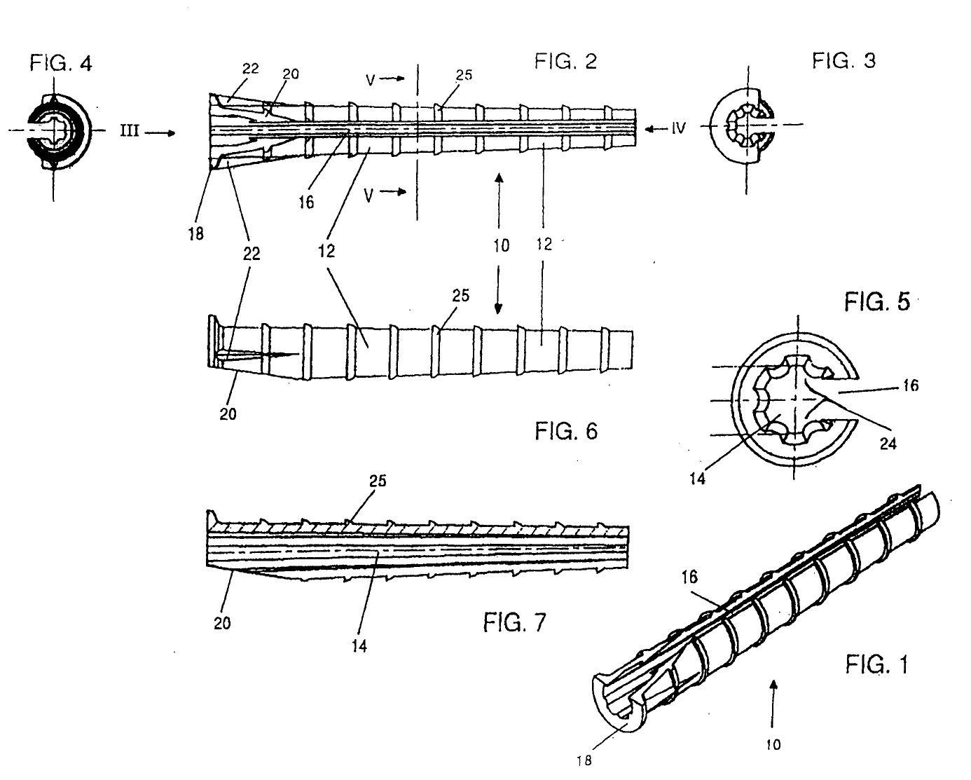

FIG. 1 shows a perspective view of a bone dowel according to the invention;

FIG. 2 shows a view of the bone dowel, seen in the direction of the plane of the slit;

FIG. 3 is an end view of the dowel in the direction of the arrow III in FIG. 2;

FIG. 4 is an end view of the dowel in the direction of the arrow IV in FIG. 2;

FIG. 5 is a section along the line V-V in FIG. 2, shown on a larger scale;

FIG. 6 is a view of the dowel according to FIG. 2, turned through 90°;

FIG. 7 is an axial section through the bone dowel according to FIGS. 1 through 6;

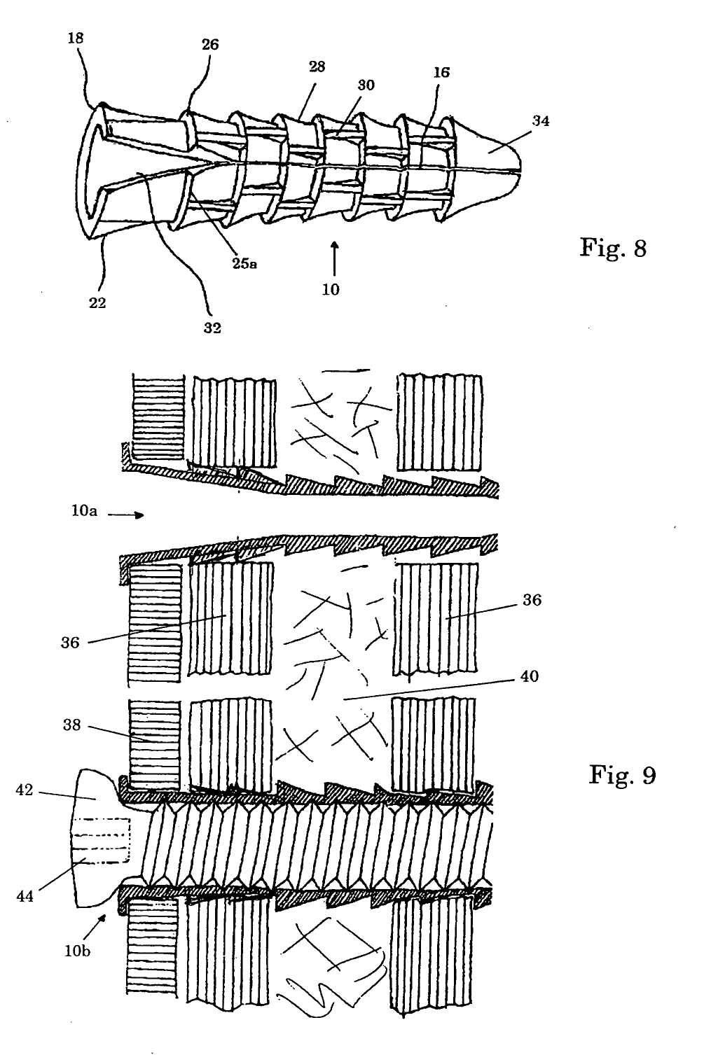

FIG. 8 is a perspective view of a preferred illustrative embodiment of a bone dowel according to the invention;

FIG. 9 is a sectional view of dowels anchored in the bone and holding an osteosynthesis plate.

The bone dowel 10 according to FIGS. 1 through 7 consists of a dowel jacket 12 which is circular in cross section, gently conical on the outside, encloses a cylindrical through-hole 14, and has a continuous longitudinal slit 16 extending over a generating line. The external diameter of the dowel jacket 12 decreases from the head part toward the dowel base, as a result of which the insertion into the bore in the bone is made easier. With an external diameter of 5 mm in the head part, the longitudinal slit 16 has a width of approximately 1.5 mm. The through-hole 14 enclosed by the dowel jacket 12 has a diameter of approximately 4 mm. At the head end, the dowel jacket 12 is shaped to form a head flange 18 in the manner of a countersunk head. At the head end, the dowel jacket 12 has a bevel 20 which at the center is passed through by the slit 16. This bevel extends from the head flange toward the outer circumference of the dowel jacket, so that the width of the bevel, as can be seen from FIG. 2, decreases from the head flange 18. On the outer surface, in the head part, the dowel jacket has means securing it against rotation, these means being in the form of three longitudinal ribs 22 (fins) which are arranged at an angle of 120° from one another and whose height decreases from the radially external diameter of the head flange 18 toward the dowel jacket and merges into the jacket. The cross section of the longitudinal ribs has the shape of a sharp-edged isosceles triangle.

On the inside, the dowel jacket 12 has webs 24 which extend continuously along its length and have the cross section of a segment of a circle.

As can be seen from the drawing, the dowel jacket 12 has, on its outside, annular ribs 25 which are arranged at an axial distance from one another, extend in the circumferential direction, and are formed with sharp edges. In particular, these annular ribs 25 are configured like barbs with a steep leading flank 26 pointing toward the dowel head, and with a gentle trailing flank 28 extending toward the dowel jacket. In this way, it is possible to reliably avoid an axial displacement inside the bore in the bone during and after insertion of the screw, but the gentle flanks nevertheless permit easy insertion, while the steep flank of the ribs prevents pulling out.

The illustrative embodiment according to FIG. 8 corresponds to the illustrative embodiment according to FIGS. 1 through 7, with the difference that the annular ribs 25a are shaped conically in such a way that their gentle trailing flanks 28 in each case slope down as far as the steep leading flanks 26 of the annular ribs. The trailing flanks 28 have longitudinal webs 30 whose height corresponds to the maximum height of the annular ribs 25a and which in each case are offset in the circumferential direction relative to the longitudinal webs of the trailing flanks 28 following in the axial direction. The bevel 32 in this illustrative embodiment is designed as a V-shaped inlet aperture which opens symmetrically into the longitudinal slit 16. The dowel has a dome-shaped dowel base 34.

The head flange 18 is used to bear on the plate surface, thus ensuring that the dowel does not slip into the plate hole. The longitudinal ribs 22 in the form of fins are used for wedging in the hole. The beveled V-shaped inlet aperture 32 permits direct contact of a screw thread section and in this way avoids the tendency of the dowel to rotate in the screw hole in the plate. After the screw has been screwed in, the annular ribs 25a are able to hook in behind the cortical substance on either side and thus improve the screw hold. When the screw is screwed in, the longitudinal webs 30 are pressed against the cortical substance and, together with the longitudinal ribs 22 and the beveled inlet aperture 32, prevent rotation of the dowel in the bone. The longitudinal slits 16 permit spreading-open of the dowel. The conical dome-shaped dowel tip 34 makes insertion of the dowel easier.

FIG. 9 shows the anchoring of a bone dowel according to FIG. 8 in the cortical substance 36 for the purpose of securing a metal osteosynthesis plate 38. As can be seen from FIG. 9, the dowel penetrates the spongy substance 40. In the drawing, the inserted bone dowel 10a is shown prior to insertion of the screw, and the bone dowel 10b is shown in the spread-open state, the spreading being effected by a screw 42 which has been tightened via a hexagon socket 44.

As can be seen, the barb-shaped annular ribs are able to deflect radially upon insertion of the bone dowel into the pre-drilled hole and, after spreading, these annular ribs provide a reliable hold against slipping out.

The bone dowels shown in the drawing can be made of a plastic suitable for surgical purposes or of an absorbable material. It is also possible for the dowel and/or screw to be made of stainless steel or of another suitable metal, e.g. of titanium. By virtue of the longitudinal slit, the dowel is able to reduce in diameter upon insertion into the bore in the bone, which, in conjunction with the gentle flanks of the circumferential ribs, makes insertion easier. Withdrawal, with or without an inserted screw, is made difficult by the steep flank of the barb.

On completion of the bone-healing process, the dowel can be removed together with the screw if these are made of non-absorbable material. If the bone dowel and/or the screw are made of absorbable material, these will already have been absorbed during the bone-healing process and replaced by endogenous bone tissue and thus remain in the body. Dowels and/or screws made of metal can be removed or, for medical considerations, can also be left in the body.

### LIST OF REFERENCE NUMBERS

10 bone dowel
12 dowel jacket
14 through-hole
16 longitudinal slit
18 head flange
20 bevel
22 longitudinal ribs
24 webs
25 annular ribs
25a annular ribs
26 leading flank
28 trailing flank
30 longitudinal webs
32 bevel, V-shaped inlet aperture
34 dome-shaped dowel base
36 cortical substance
38 osteosynthesis plate
40 spongy substance
42 screw
44 hexagon socket

1. A bone dowel (10) for inserting a screw, for example for mutual fixing of bone fragments by means of an osteosynthesis plate, having the following features:
a dowel jacket (12) of circular cross section, gently conical on the outside, tapering from the dowel head to the dowel base, has a through-hole (14) with a uniform cross section along its length;
the dowel jacket (12) is interrupted along a generating line by a longitudinal slit (16), by which means the dowel jacket acquires a continuously C-shaped cross section;
a limiting head flange (18) in the form of a countersunk head is formed integrally on the head part;
the head part of the dowel jacket is provided, in the area of the longitudinal slit (16), with a bevel whose width decreases from the head part toward the dowel jacket;
the dowel jacket has annular ribs (25) distributed with axial spacings along its entire length.

2. The bone dowel (10) as claimed in claim 1, wherein the annular ribs (25) are designed with sharp edges and in the form of barbs, and they have a steep flank (26) directed toward the head part, and a gentle flank (28) directed toward the base part.

3. The bone dowel (10) as claimed in claim 1, in which the dowel jacket additionally has longitudinal ribs (22) and longitudinal webs (30) as means of securing against rotation.

4. The bone dowel (10) as claimed in claim 1, in which the longitudinal webs (30) each extend between the annular ribs (25a), their outer edge extending in the maximum radial height of the annular ribs.

5. The bone dowel (10) as claimed in claim 4, in which the axially adjacent longitudinal webs (30) are each mutually offset in the circumferential direction.

6. The bone dowel (10) as claimed in claim 2, in which the gentle trailing flanks (28) directed toward the base part are of cone-shaped configuration and each extend as far as the steep leading flank (26) of the following annular rib (25a).

7. The bone dowel (10) as claimed in claim 1, in which the bevel is configured as a V-shaped inlet aperture (32) of the longitudinal slit (16).

8. The bone dowel (10) as claimed in claim 1, in which, in the head part, the dowel jacket has longitudinal ribs (22) whose height decreases from the head flange (18) toward the first transverse rib.

9. The bone dowel (10) as claimed in claim 1, in which the last annular rib in the base part merges into a dome-shaped dowel base (34).

10. The bone dowel (10) as claimed in claim 1, in which the dowel and/or the screw is made of absorbable material.
